SEQUENCE LISTING (3 sequences)

SEQ ID NO: 1 - RNA, 1561 nt, synthetic construct
  source 1..1561: mol_type = other RNA
attgagagtttgatcctggctcaggacgaacgctggcggcgtgcctaatacatgcaagtcgagcgagctgaattcaaagatcccttcggggtgatttgttggacgctagcggcggatgggtgagtaacacgtgggcaatctgccctaaagactgggataccacttggaaacaggtgctaataccggataacaacatgaatcgcatgattcaagtttgaaaggcggcgcaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggtcttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaactccatgtgtagcggtggaatgcgtagatatatggaggaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgcgctacacctagagataggtggttcccctcggggacgcagagacaggtggtgcatggctgtcgtcagctcgtgtcgtgagatgttgggttaagtcccgcaacgagcgcaacccttgtctttagttgccatcattaagttgggcactctaaagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgctctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgagataacctttataggagtcagccgcctaaggcagggcagatgactggggtgaagtcgtaacaaggtagccgtaggagaacctgcggctggatcacctccttt

SEQ ID NO: 2 - RNA, 1455 nt, synthetic construct
  source 1..1455: mol_type = other RNA
gctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaagtgaggctcgtgattgggactaa

SEQ ID NO: 3 - RNA, 1417 nt, synthetic construct
  source 1..1417: mol_type = other RNA
gctcaggatgaacgctggcggcgtgcttaacacatgcaagtcgaacgggatccaggcagcttgctgcctggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaacccctt

(19) United States
(12) Patent Application Publication
(10) Pub. No.: US 2026/0053869 A1
(43) Pub. Date: Feb. 26, 2026

(54) LACTOBACILLUS DELBRUECKII SUBSP. LACTIS CKDB001 STRAIN, AND COMPOSITION FOR PREVENTION, AMELIORATION, OR TREATMENT OF NON-ALCOHOLIC FATTY LIVER COMPRISING SAME

(71) Applicant: CKD BIO CORPORATION, Seoul (KR)

(72) Inventors: Hyunchae JOUNG, Gyeonggi-do (KR); Byoung-Kook KIM, Seoul (KR); Bo Hye HEO, Gyeonggi-do (KR); Min Ho SEO, Gyeonggi-do (KR); Woo Ri KIM, Incheon (KR)

(21) Appl. No.: 19/384,429

(22) Filed: Nov. 10, 2025

Related U.S. Application Data
(62) Division of application No. 17/913,210.

(30) Foreign Application Priority Data
Mar. 23, 2020 (KR) 10-2020-0034823

(51) Int. Cl. A61K 35/747; A23L 33/135; A61K 35/745; A61P 1/16; C12N 1/20; C12R 1/225

(52) U.S. Cl. CPC A61K 35/747; A23L 33/135; A61K 35/745; A61P 1/16; C12N 1/20; C12R 2001/225

(57) ABSTRACT

The present invention relates to a Lactobacillus lactis CKDB001 strain, and a composition for the prevention or treatment of non-alcoholic fatty liver comprising same. When the composition of the present invention is ingested, the composition has the effect of ameliorating high-fat diet-induced fatty cirrhosis and inflammations caused thereby, and thus the composition of the present invention can be effectively used as a preparation for ameliorating or treating non-alcoholic fatty cirrhosis and steatohepatitis.

### TECHNICAL FIELD

The present disclosure has been made with the support of the Ministry of Science and ICT of the Republic of Korea under Project No. 2018M3A9F3020942, which was conducted by Hallym University in the research program named “Development of pharmabiotics for chronic liver disease based on research into gut microbiome” as a branch of the research project titled “Microorganism control and applied source technology development”, under the research management of the National Research Foundation of Korea, from 1 Apr. 2018 to 31 Dec. 2022.

This patent application claims the benefit of and priority to Korean Patent Application No. 10-2020-0034823 filed with the Korean Intellectual Property Office on Mar. 23, 2020, the disclosure of which is incorporated herein by reference.

The present disclosure relates to a Lactobacillus delbrueckii subsp. lactis CKDB001 (Lactobacillus lactis CKDB001) strain and a composition comprising same for prevention or treatment of non-alcoholic fatty liver.

### BACKGROUND ART

Nonalcoholic fatty liver disease (NAFLD) is one of the most common liver diseases worldwide due to the effects of metabolic syndrome on liver metabolism. The incidence of obesity and related diseases, including NAFLD, has increased rapidly in all age groups in the world. NAFLD includes non-alcoholic steatohepatitis (NASH), which can progress from pure fatty steatosis (fat infiltrates more than 5% of hepatocytes) to cirrhosis, liver failure, and hepatocellular carcinoma (HCC). Within the pathophysiology of NAFLD, there are glucose and lipid metabolism, genetic predisposition, environmental fat accumulation, and intestinal microbial factors. The risk of death from liver disease is remarkably increased in progressive fibrosis patients suffering from bridging fibrosis or cirrhosis. Recent advances in knowledge and technology about the human gut microbiota have changed our perspective on human disease and treatment. The human gut microbiota is a complex ecosystem composed of various bacteria with a total mass of about 1-2 kg. The gut microbiota maintains a close relationship with the host and plays an important role in vitamin production, the mucosal immune system, and bacterial translocation. The intestine is connected directly to the liver via the portal vein. The liver is also referred to as “gut-liver axis” as a major metabolic organ of gut products composed of important dietary nutrients and microbe-related components. Disturbance of the gut microbiome may lead to liver disease, including NAFLD. Conventional therapies for NAFLD are often ineffective due to poor compliance, lack of efficacy, or changes in diet and lifestyle. Theoretically, the regulation of gut microbiota through the administration of probiotics can be effective, and recent studies have revealed several possibilities to support this theory. Lactobacillus delbrueckii subsp. lactis is known as a beneficial strain capable of alleviating various diseases including enteritis and inflammatory and metabolic diseases. In the present disclosure, Lactobacillus delbrueckii subsp. lactis and Bifidobacterium strains and a combination thereof were evaluated for therapeutic effects in NAFLD mouse models.

### DISCLOSURE OF INVENTION

### Technical Problem

Leading to the present disclosure, intensive and thorough research conducted by the present inventors into the development of a lactic acid agent for prevention or treatment of non-alcoholic fatty liver disease resulted in isolating and identifying a Lactobacillus delbrueckii subsp. lactis CKDB001 strain and finding that a composite composition of the same strain and Bifidobacterium remarkably lowers the incidence of non-alcoholic fatty liver disease and inflammation.

Therefore, an aspect of the present disclosure is to provide a Lactobacillus delbrueckii subsp. lactis CKDB001 strain.

Another aspect of the present disclosure is to provide a composition containing a Lactobacillus delbrueckii subsp. lactis CKDB001 strain for prevention or treatment of non-alcoholic fatty liver disease.

A further aspect of the present disclosure is to provide a food composition containing a Lactobacillus delbrueckii subsp. lactis CKDB001 strain for prevention or alleviation of non-alcoholic fatty liver disease.

### Solution to Problem

According to an aspect thereof, the present disclosure provides a Lactobacillus delbrueckii subsp. lactis CKDB001 strain with accession no. KCTC 14149BP.

According to another aspect thereof, the present disclosure provides a composition containing a Lactobacillus delbrueckii subsp. lactis CKDB001 strain with accession number KCTC 14149BP.

According to a further aspect thereof, the present disclosure provides a pharmaceutical composition containing a Lactobacillus delbrueckii subsp. lactis CKDB001 strain with accession no. KCTC 14149BP as an active ingredient for prevention or treatment of fatty liver disease.

According to another aspect thereof, the present

disclosure provides a food composition containing Lactobacillus delbrueckii subsp. lactis CKDB001 strain with accession no. KCTC 14149BP as an active ingredient for prevention or alleviation of fatty liver disease.

The present inventors isolated a new strain which was named a Lactobacillus delbrueckii subsp. lactis CKDB001 strain with accession no. KCTC 14149BP. In addition, the present inventors prepared a lyophilizate powdered formulation with the strain and a mixture of a Bifidobacterium sp. strain therewith. The powdered formulation prepared with the strain or the mixed strains has a palliative or therapeutic effect on non-alcoholic fatty liver disease as it reduced steatosis and inflammation in test animal models of high-fat diet-induced non-alcoholic fatty liver disease when administered thereto.

As used herein, the term “fatty liver” refers to a case of fat deposition in the liver with neither hepatocellular damage (ballooning degeneration) nor fibrosis. The term “steatohepatitis” refers to a case of inflammatory findings accompanied by hepatocyte damage (balloon degeneration) while showing fat deposition in the liver. Steatohepatitis may be accompanied with liver fibrosis. The term “liver cirrhosis” means liver cirrhosis accompanied by histological findings of fatty liver or steatohepatitis, or liver cirrhosis occurring in a patient with fatty liver or steatohepatitis that has been histologically proven. Herein, definitions of the terms related to fatty liver disease are only intended to include various conditions related to fatty liver, and the conditions of patients defined according to the terms may not always be clearly distinguished.

In an embodiment of the present disclosure, the fatty liver is non-alcoholic fatty liver disease or alcoholic fatty liver disease.

As used herein, the term “non-alcoholic fatty liver disease” (NAFLD) refers to fatty liver disease caused by factors other than alcohol. The term “non-alcoholic fatty liver disease” is intended to encompass “non-alcoholic fatty liver disease”, “non-alcoholic steatohepatitis”, “non-alcoholic fatty liver-associated fibrosis”, and “non-alcoholic fatty liver-associated cirrhosis”, which are all in hepatocytic fat accumulated states due to factors other than alcohol.

In the present disclosure, the Lactobacillus delbrueckii subsp. lactis CKDB001 was deposited with the Korean Collection for Type Culture (KCTC) under accession n. KCTC 14149BP. Hereinafter the Lactobacillus delbrueckii subsp. lactis CKDB001 is abbreviated to Lactobacillus lactis or Lactobacillus lactis strain CKDB001.

Cell states of the Lactobacillus delbrueckii subsp. lactis CKDB001 strain are as follows:
1) Bacterial morphology: Gram-positive rod or coccobacillus
2) Physiological properties: The bacterium is a facultative anaerobe that optimally grows at a temperature of 36-38° C. and a pH of 5.5-5.8 and is asporogenous without motility.
3) It is generally isolated from fermented milk or cheese and considered safe for use in foods (probiotics and dairy products).

In an embodiment of the present disclosure, the composition further contains a Bifidobacterium sp. strain as an active ingredient.

Examples of the Bifidobacterium sp. strain include, but are not limited to, Bifidobacterium bifidum, Bifidobacterium lactis, Bifidobacterium animalis lactis, Bifidobacterium longum, Bifidobacterium breve, and the like, more specifically, Bifidobacterium longum and Bifidobacterium breve.

In a particular embodiment of the present disclosure, the Bifidobacterium longum is a Bifidobacterium longum CKDB004 strain, and is deposited with the KCTC under accession number “KCTC 13671BP”.

In another particular embodiment of the present disclosure, the Bifidobacterium breve is a Bifidobacterium breve CKDB002 strain, and is deposited with the KCTC under accession number “KCTC 13893BP”.

The cell states of the Bifidobacterium longum strain are as follows:
1) Bacterial morphology: Gram-positive, amorphous rod-shaped
2) Physiological properties: The bacterium is an absolute anaerobe with an optimal growth temperature of 36-38° C. and an optimal pH of 6.5-7.0. It lacks motility and gelatin liquefaction and cannot produce endospores.
3) It is isolated in the human digestive system and is most often found and isolated in the intestines of newborns who have been breastfed after birth. It is considered safe for use in foods (probiotics and dairy products).

The cell states of the Bifidobacterium breve strain are as follows:
1) Bacterial morphology: Gram-positive, amorphous rod-shaped
2) The bacterium is an absolute anaerobe with an optimum growth temperature of 36-38° C. and an optimum pH of 6.5-7.0. It lacks motility and gelatin liquefaction and cannot produce endospores.
3) It is isolated from the human digestive system and is most often found in the intestines of newborns who have been breastfed after birth. It is considered safe for use in foods (probiotics and dairy products).

The biochemical properties of the aforementioned strains of the present disclosure are given in Table 1 below, and the 16s rRNA sequences of each strain are shown in SEQ ID NO: 1 to SEQ ID NO: 3.

TABLE 1

| | | L. lactis | B. longum | B. breve |
|---|---|---|---|---|
| Source | | CKDB001 | CKDB004 | CKDB002 |
| Temoin | 0 | + | + | + |
| Glycerol | 1 | − | − | − |
| Erythritol | 2 | − | − | − |
| D-Arabinose | 3 | − | − | − |
| L-Arabinose | 4 | − | + | − |
| D-Ribose | 5 | − | − | − |
| D-Xylose | 6 | − | + | − |
| L-Xylose | 7 | − | − | − |
| D-Adonitol | 8 | − | − | − |
| Methyl-lolXylopyranoside | 9 | − | − | − |
| D-Galactose | 10 | − | + | + |
| D-Glucose | 11 | + | + | − |
| D-Fructose | 12 | + | + | − |
| D-Mannose | 13 | + | − | + |
| L-Sorbose | 14 | − | − | − |
| L-Rhamnose | 15 | − | − | − |
| Dulcitol | 16 | − | − | − |
| Inositol | 17 | − | − | − |
| D-Mannitol | 18 | − | + | − |
| D-Sorbitol | 19 | − | + | − |
| Methyl-tolMannopyranoside | 20 | − | − | − |
| Methyl-ranGlucopyranoside | 21 | − | − | − |
| N-Acetylglucosamine | 22 | + | − | + |
| Amygdaline | 23 | − | − | − |
| Arbutine | 24 | − | − | − |
| Esculine, citrate de fer | 25 | − | − | − |
| Salicine | 26 | − | + | − |
| D-Cellobiose | 27 | − | − | − |
| D-Maltose | 28 | w | + | + |
| D-Lactose | 29 | + | + | + |
| D-Melibiose | 30 | − | + | + |
| D-Saccharose | 31 | + | + | + |
| D-Trehalose | 32 | + | − | − |
| Inuline | 33 | − | − | − |
| D-Melezitose | 34 | − | − | − |
| D-Raffinose | 35 | − | + | + |
| Amidon | 36 | − | − | − |
| Glycogene | 37 | − | − | − |
| Xylitol | 38 | − | − | − |
| Gentiobiose | 39 | − | − | + |
| D-Turanose | 40 | − | + | − |
| D-Lyxose | 41 | − | − | − |
| D-Tagatose | 42 | − | − | − |
| D-Fucose | 43 | − | − | − |
| L-Fucose | 44 | − | − | − |
| D-Arabitol | 45 | − | − | − |
| L-Arabitol | 46 | − | − | − |
| Potassium GlucoNaTe | 47 | − | − | − |
| Potassium 2-CetoGluconate | 48 | − | − | − |
| Potassium 5-CetoGluconate | 49 | − | − | − |

When used as an active ingredient in the compositions of the present disclosure, the strain is intended to encompass an isolated and/or purified cell mass thereof, a cell culture including the cell mass, an extract from the cell mass, a supernatant of the culture, a concentrated liquid, concentrate, or dried substance thereof, a dilution or a diluted substance thereof as necessary, and any product obtained by processing the cell culture or medium.

The cell culture method, extraction method, separation method, concentration method, drying method, dilution method, etc. are not particularly limited.

A medium for culturing the cells typically contains milk proteins such as skim milk, whey, and casein, sugars, yeast extracts, etc., and various general aerobic or anaerobic methods can be appropriately used as the culture method.

Also useful is a neutralization culture method in which the culture temperature is set to be, for example, 30 to 40° C. and the medium is maintained at a neutral to acidic state, for example, at a pH of 5 to 6 using alkali such as sodium hydroxide during cultivation. In addition to the neutralization culture method, any culture method such as a batch culture method may be used. After culturing, if necessary, the culture or its supernatant may be concentrated, dried, diluted, or the like.

Furthermore, a cell culture is separated by centrifugation or through a membrane into a supernatant and a cell mass which may be concentrated and recovered as a concentrate. Moreover, the cell mass may be subjected to ultrasonication or enzymatic treatment to extract the components in the cell mass. In an alternative, the culture or the supernatant thereof, or the cells mass or the extract thereof may be dried. These matters can be used as an active ingredient in the composition of the present disclosure.

When the composition of the present disclosure is prepared as a food composition, it may include an additive commonly used in food production as well as the lactic acid bacteria as an active ingredient. Examples of the additive includes proteins, carbohydrates, fats, nutrients, seasonings and flavoring agents. The carbohydrates may be common sugars, such as monosaccharides (e.g., glucose, fructose, etc.), disaccharides (e.g., maltose, sucrose, oligosaccharides, etc.), and polysaccharides (e.g., dextrin, cyclodextrin, etc.), and sugar alcohols, such as xylitol, sorbitol, erythritol, and the like. As flavoring agents, natural flavoring agents (taumatin, stevia extract (e.g., rebaudioside A, glycyrrhizin, etc.)) and synthetic flavoring agents (saccharin, aspartame, etc.) can be used.

For instance, when the food composition of the present disclosure is prepared as a drink, citric acid, high fructose corn syrup, sugar, glucose, acetic acid, malic acid, fruit juice, a jujube extract, or a licorice extract may be additionally included in addition to the strain as an active ingredient of the present disclosure.

The food composition of the present disclosure includes processed forms of all natural materials, such as foods, functional foods, nutritional supplements, health foods, and food additives. These types of food compositions can be prepared in various forms according to conventional methods known in the art.

For health foods, for example, the lactic acid bacteria themselves may be prepared into beverage forms, such as tea, juices, and drinks, or into granules, capsules, and powders. As foods, beverages (inclusive of alcoholic beverages), fruits and processed foods thereof (e.g., canned fruits, bottled foods, jam, marmalade, etc.), fishes, meats, and processed foods thereof (e.g., ham, sausage corn beef, etc.), breads and noodles (e.g., udon noodles, soba noodles, ramen, spaghetti, macaroni, etc.), fruit juices, various drinks, cookies, syrup, dairy products (e.g., yogurt, fermented milk, butter, cheese, etc.), edible vegetable oils and fats, margarine, vegetable proteins, retort foods, frozen foods, various seasonings (e.g., soybean paste, soy sauce, sauce, etc.) can be added with the lactic acid bacteria of the present disclosure. Furthermore, for use in the form of a food additive, the lactic acid bacteria of the present disclosure may be prepared into powders or concentrated liquids.

When the composition of the present disclosure is prepared as a pharmaceutical composition, the pharmaceutical composition includes a pharmaceutically acceptable carrier. So long as it is commonly used for typical formulations, any pharmaceutically acceptable carrier is available in the pharmaceutical composition of the present disclosure. Examples of the pharmaceutically acceptable carrier include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate and mineral oil, but with no limitations thereto. The pharmaceutical composition of the present disclosure may further include a lubricant, a humectant, a sweetener, a flavoring agent, an emulsifier, a suspending agent, a preservative, and the like, in addition to the above components.

The pharmaceutical composition of the present disclosure may be administered orally or parenterally and is preferably applied by oral administration. The pharmaceutical composition of the present disclosure may be formulated in the following various oral or parenteral dosage forms, but is not limited thereto.

Formulations for oral administration include, for example, tablets, pills, hard/soft capsules, solutions, suspensions, emulsions, syrups, granules, elixirs, and the like. In these formulations, one or more common diluents or excipients such as fillers, extenders, wetting agents, disintegrants, lubricants, binders, and surfactants may be used in addition to the active ingredient. The disintegrant may be exemplified by agar, starch, alginic acid or sodium salt thereof, calcium monohydrogen phosphate anhydride, etc. Available the lubricant include are silica, talc, stearic acid or a magnesium or calcium salt thereof, polyethylene glycol, etc. Examples of the binder include magnesium aluminum silicate, starch paste, gelatin, tragacanth, methyl cellulose, sodium carboxymethyl cellulose, polyvinyl pyrrolidine, and low-substituted hydroxypropyl cellulose. Lactose, dextrose, sucrose, mannitol, sorbitol, cellulose, or glycine, may be used as a diluent. In some cases, commonly known azeotropes, absorbents, colorants, flavorants, sweeteners, and the like may be used together.

The composition may be sterilized or contain supplements such as preservatives, stabilizers, wetting agents or emulsification accelerators, salts for regulating osmotic pressures, buffers, etc., and other therapeutically useful substances, and may be formulated according to a typical method, such as mixing, granulation, or coating methods.

A suitable dose of the pharmaceutical composition of the present disclosure may vary depending on various factors including formulation methods, administration modes, the patient's age, body weight, and sex, the severity of disease symptoms, the duration of administration, the route of administration, the excretion rate, and response sensitivity.

In an embodiment of the present disclosure, the total lactic acid bacteria in the composition may be administered at a dose of 1×10⁷-1×10¹², 1×10⁸-1×10¹², 1×10⁹-1×10¹², 1×10¹⁰-1×10¹², 1×10⁷-1×10¹¹, 1×10⁷-1×10¹⁰, 1×10⁷-1×10⁹, 1×10⁷, 1×10⁸, 1×10⁹, 1×10¹⁰, or 1×10¹¹ cfu/day, but with no limitations thereto.

The pharmaceutical composition of the present disclosure may be formulated with a pharmaceutically acceptable carrier and/or excipient and prepared into a unit dosage form or enclosed in a multiple-dose container according to a method that could be easily performed by a person skilled in the art to which the present disclosure pertains. In this regard, the formulation may be in the form of a solution, suspension, or emulsion in an oily or aqueous medium, or an extract, pulvis, powders, granules, tablets, or capsules, and may additionally include a dispersant or a stabilizer.

According to another aspect thereof, the present disclosure provides a method for prevention or treatment of fatty liver disease, the method comprising a step of administering to a subject a pharmaceutical composition containing Lactobacillus delbrueckii subsp. lactis CKDB001 with accession no. KCTC 14149BP as an active ingredient for prevention or treatment of non-alcoholic fatty liver disease.

As used herein, the term “administration” or “administering” refers to directly administering a therapeutically or prophylactically effective amount of the composition of the present disclosure to a subject (individual) suffering from or likely to suffer from the target disease to form the same amount in the body of the subject.

The term “therapeutically effective amount” of the composition, as used herein, means an amount of the composition sufficient to provide a therapeutic or prophylactic effect to a subject to which the composition is administered, and thus is intended to encompass a “prophylactically effective amount”.

In addition, as used herein, the term “subject (individual)” refers to mammals, including humans, mice, rats, guinea pigs, dogs, cats, horses, cattle, pigs, monkeys, chimpanzees, baboons and rhesus monkeys. Most specifically, the subject of the present disclosure is a human.

Since the method for preventing or treating fatty liver disease according to the present disclosure includes administering the pharmaceutical composition according to an aspect of the present disclosure, the overlapping description therebetween is omitted in order to avoid overdue complexity of the present specification.

### Advantageous Effects of Invention

The present disclosure provides a Lactobacillus lactis CKDB001 stain, a composition containing the strain and/or a Bifidobacterium strain for prevention, alleviation, or treatment of non-alcoholic fatty liver. When ingested, the composition of the present disclosure has an effect of alleviating high-fat diet-induced fatty liver disease and the resulting inflammation. Thus, the composition of the present disclosure can find advantageous applications as an agent for alleviation or treatment of non-alcoholic fatty liver disease and steatohepatitis.

### BRIEF DESCRIPTION OF DRAWINGS

FIGS. 1a and 1b are schematic views of designs for experiment methods of the present disclosure.

FIGS. 2a and 2b are graphs of liver/body weight ratios of mice after 9 weeks of administration of single or composite strains of the present disclosure while feeding a normal diet or a western diet thereto.

FIGS. 3a and 3b and FIGS. 4a and 4b are graphs of histopathological examination results of mice after 9 weeks of administration of single or composite strains of the present disclosure while feeding a normal diet or a western diet thereto (FIGS. 3a and 3b: fat infiltration, FIGS. 4a and 4b: inflammation).

FIGS. 5a and 5b and FIGS. 6a and 6b are graphs of levels of liver enzymes (AST and ALT) in blood of mice after 9 weeks of single or composite strains of the present disclosure while feeding a normal diet or a western diet thereto (FIGS. 5a and 5b: AST, FIGS. 6a and 6b: ALT).

FIGS. 7a, 7b and 7c are graphs of mRNA and protein expression levels of inflammatory cytokines of mice after 9 weeks of single or composite strains of the present disclosure while feeding a normal diet or a western diet thereto (FIG. 7a: cytokine mRNA expression levels, FIGS. 7b and 7c: cytokine protein expression levels).

FIG. 8 is a view showing protein expression levels of occludin, a main tight junction constituent of intestinal epithelial cells in mice after 9 weeks of single or composite strains of the present disclosure while feeding a normal diet or a western diet thereto.

### MODE FOR CARRYING OUT THE INVENTION

A better understanding of the present disclosure may be obtained through the following examples which are set forth to illustrate, but are not to be construed to limit, the present disclosure.

### EXAMPLES

Throughout this specification, “%” used to indicate the concentration of a specific substance is (weight/weight) % for solid/solid, (weight/volume) % for solid/liquid, and (volume/volume) % for liquid/liquid.

### Strain Used

The probiotics used in this experiment were isolated from fermented milk, newborn feces, and healthy adult feces. A list of strains and sources used in the present disclosure is given in Table 2.

TABLE 2

| No. | Stain | Abbreviated | Source |
|---|---|---|---|
| 1 | Lactobacillus delbrueckii | LL | Raw milk |
| | subsp. lactis CKDB001 | | fermentate |
| 2 | Bifidobacterium longum CKDB004 | BLO | Healthy |
| | | | adult feces |
| 3 | Bifidobacterium breve CKDB002 | BBR | Newborn feces |
| 4 | Lactobacillus rhamnosus GG | LGG | — |

### Probiotics Preparation

Seed culture was performed at 37° C. for 24 hours in a flask containing MRS or BL broth for lactic acid bacteria seeds. Each culture was inoculated into an optimized medium in a fermenter (Bio Control & Science, MARADO-05D-PS). Fermentation was carried out at 37° C. for 18-20 hours while stirring at 120 rpm during which the pH was maintained constant at 5.5-6.0 by automatically adding a NaOH solution (25% w/v) to the medium. Lyophilization of 40× enriched cells was performed according to the manual (Cooling & Heating System, Lab-Mast 10). After lyophilization, colony-forming units (CFU) per 1 g of each probiotic powder were measured by serial dilution. Probiotics were suspended in 0.1 M PBS and adjusted to a density of 109 CFU/mL prior to use.

### Laboratory Animals

Specific-pathogen free (SPF) male C57BL/6J mice with 6 weeks of age were purchased from Dooyeol Biotech (Seoul, Korea). All mice were individually housed in steel micro-insulator cages at 22±2° C. with a light-dark cycle of 12 h/12 h. The mice had free access to water and food throughout the experiment and were monitored daily. For one week, they were acclimatized to the experimental site while being fed with a normal diet, and the group fed with a western diet (western diet or high fat diet) had an intake adaptation period of 3 days. All animals received humane treatment, and all procedures were performed in accordance with the Health Guidelines for the Care and Use of Laboratory Animals. All procedures were carried out with the approval of the Committee on Animal Care and Use of Hallym University College of Medicine.

### Experimental Design

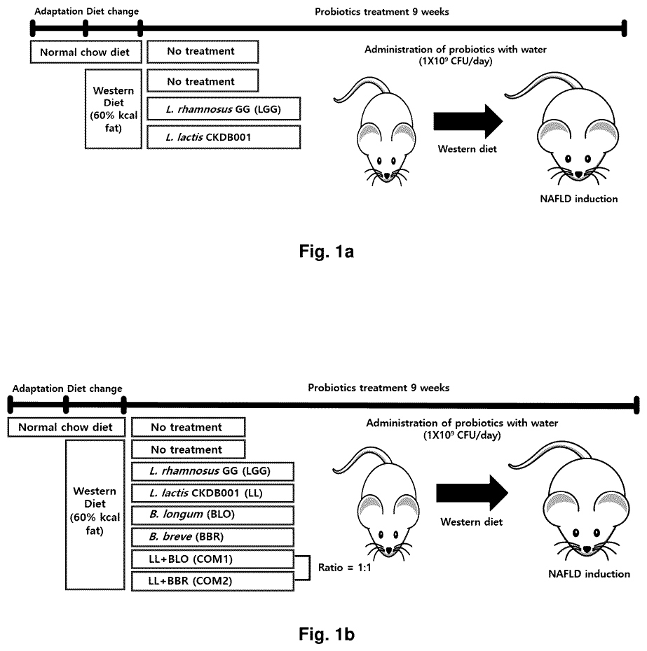

In order to examine the effects of the single and composite strains of the present disclosure on non-alcoholic fatty liver, experimental groups of 6-week-old C57BL/6 mice for the single and composite strains were designed before the experiments (see FIG. 1).

Also, examination was made of the effect of the novel strain Lactobacillus lactis CKDB001 (hereinafter expressed as CKDB001) of the present disclosure when it was administered alone. In this regard, the mice were divided into groups as follows: normal diet (ND); western diet (HFD); and western diet together with lactic acid bacteria (HFD+LGG, HFD+CKDB001) (see FIG. 1a).

In addition, the effect of the composite strain including the Lactobacillus lactis CKDB001 strain (expressed as LL) of the present disclosure was examined. In this regard, the mice were divided into groups as follows: normal diet (ND); western diet (HFD); western diet plus single lactic acid bacteria (LGG, LL, BLO, BBR; and western diet plus combined lactic acid bacteria (LL+BLO, LL+BBR) (see FIG. 1b).

Under the foregoing conditions, experiments were carried out for 9 weeks. When used alone, the lactic acid bacteria were diluted in drinking water and administered at a dose of 10⁹ CFU/day. A composite of two strains was mixed at a ratio of 1:1 and diluted in drinking water and administered at a total dose of 10⁹ CFU/day. In Examples 1 to 5 below, the experimental results of the two designs are described.

### Example 1: Liver/Body Weight Ratio (LB Ratio)

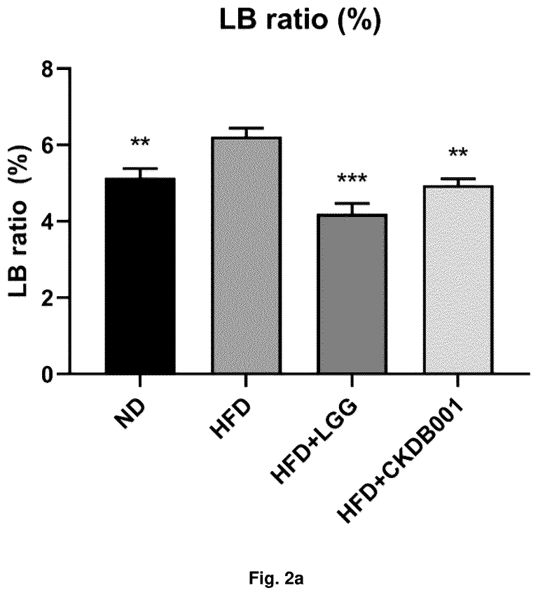

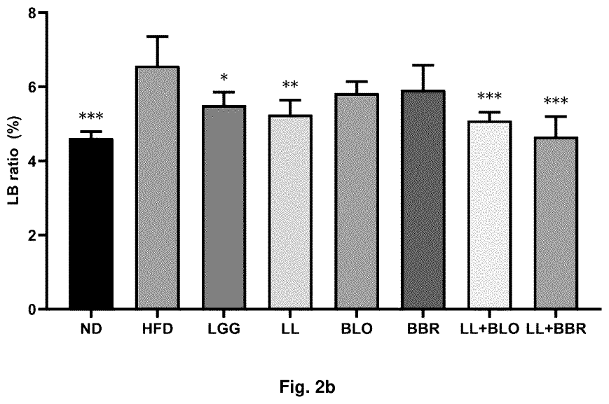

After completion of the experiments, each group of the mice was measured for body weight and liver weight to analyze liver/body weight ratios. The results are depicted in FIGS. 2a and 2b. As shown in FIG. 2a, the LB ratio was alleviated in all the single strain-administered groups, compared to the western diet-fed group. The single strain-administered groups were measured to be lower in LB ratio than the normal diet-fed group, with the superiority of the LGG strain-administered group to the CKDB001 strain-administered group of the present disclosure in terms of LB ratio improvement.

In addition, as shown in FIG. 2b, the improvement of the LB ratio was observed in all the single strain-administered groups and the composite strain-administered group, compared to the western diet group. Among others, a significant improvement in LB ratio was observed in the LL+BLO group and the LL+BBR group, each including the Lactobacillus lactis CKDB001 strain of the present disclosure.

### Example 2: Liver Histology (Steatosis and Inflammation Score)

After 9 weeks of administration, each mouse was sacrificed and the liver was taken. The liver tissue samples were fixed with 10% formalin and embedded in paraffin, followed by performing Hematoxylin and Eosin, Masson's trichrome, and reticulin stains on the liver sections.

Fatty liver (steatosis) classified by NASH Clinical Research Network Scoring System for NAFLD into grades 0-3 (0:5%, 1:5%-33%, 2:34%-66%, 3:66% or greater steatosis). Hepatitis was classified into grades 0-3 (0: no lesion, 1: 1-2 lesions per ×20 field, 2: 2-4 lesions per ×20 field, 3: more than 4 lesions per ×20 field). All biopsy specimens were analyzed by a liver pathologist.

### Steatosis Score

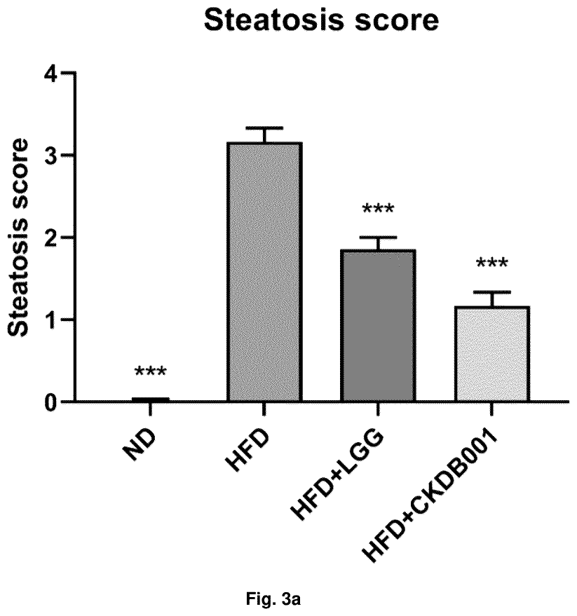

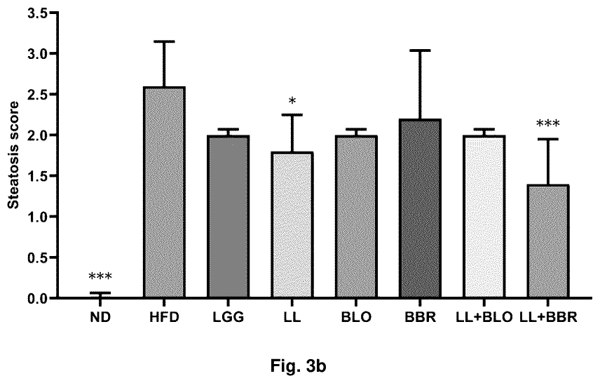

As shown in FIG. 3a, steatosis was reduced in the group fed with the single strain CKDB001 of the present disclosure, compared to the group fed with the control lactic acid strain, LGG.

In addition, as shown in FIG. 3b, liver steatosis scores were significantly reduced in all the single strain-administered groups (LGG, LL, BLO, BBR) compared to the western diet group. Among the composite strain-administered groups, a significant reduction in steatosis score was detected in the LL+BBR composite strain-administered group.

Therefore, it was found that the LL strain (CKDB001) of the present disclosure is superior to the other strains in terms of steatosis reduction, and a combination with Bifidobacterium spp., particularly with BBR brought about very excellent reduction in steatosis.

### Hepatitis Score

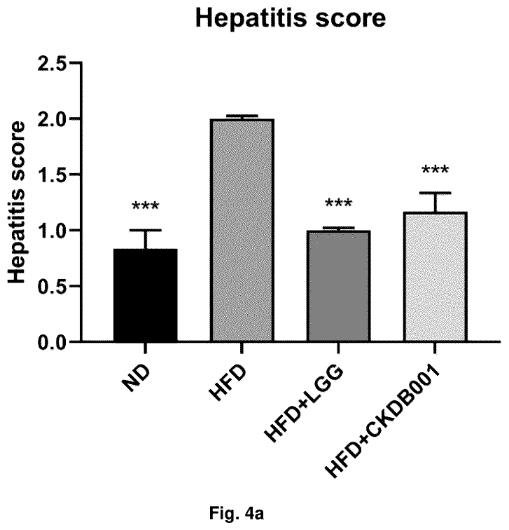

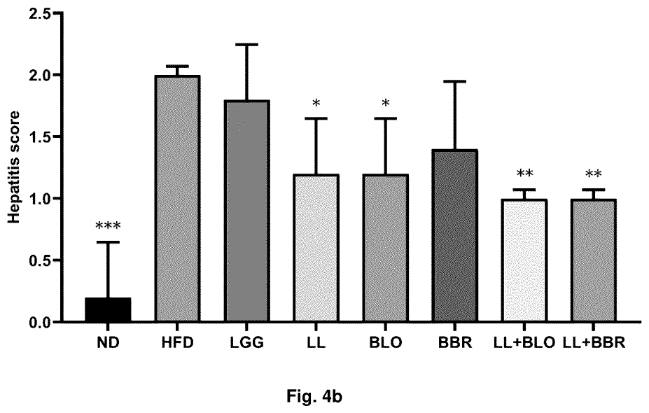

As shown in FIG. 4a, hepatitis scores in the groups fed with singe strains of the present disclosure (HFD+LGG, HFD+CKDB001) were reduced to extents similar to that of the normal diet (ND)-fed group.

In addition, as shown in FIG. 4b, all the single strain-administered groups (LGG, LL, BLO, BBR) of the present disclosure significantly decreased in hepatitis score, compared to the western diet-fed group. In particular, combinations of the LL strain of the present disclosure with Bifidobacterium sp. strains (LL+BLO, LL+BBR) exhibited synergistic effects on hepatitis score reduction, compared to the LL strain alone.

### Example 3: Liver Enzyme in Serum (AST, ALT)

Serum aspartate aminotransferase (AST) and alanine aminotransferase (ALT) were analyzed using a blood biochemistry analyzer (KoneLab 20, Thermo Fisher Scientific, Waltham, Finland).

### AST Enzyme in Blood

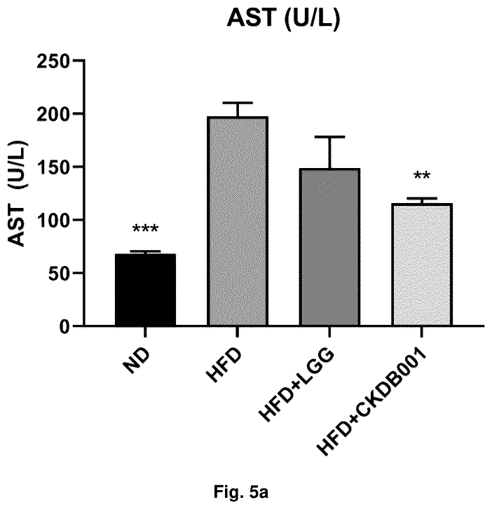

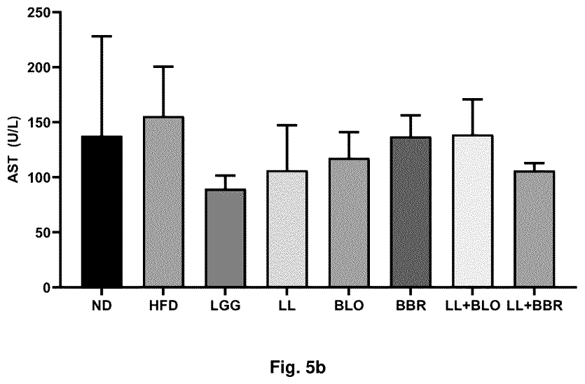

As shown in FIG. 5a, lower AST levels were detected in the groups fed with single strains, compared to the western diet-fed group (HFD), with superiority of the CKDB001 of the present disclosure to the control LGG in terms of AST reduction effect.

As shown in FIG. 5b, all of the single strain-and composite strain-administered groups has lower AST levels than that of the western diet-fed group (HFD). Among others, the greatest synergistic effect of reducing AST levels was found in the composite group of LL+BBR of the present disclosure.

### ALT Enzyme in Blood

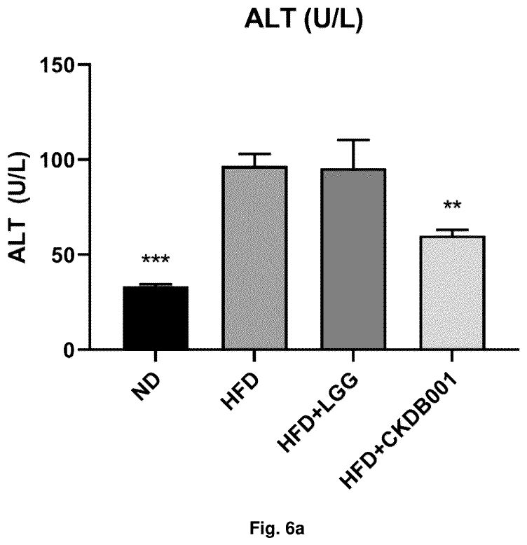

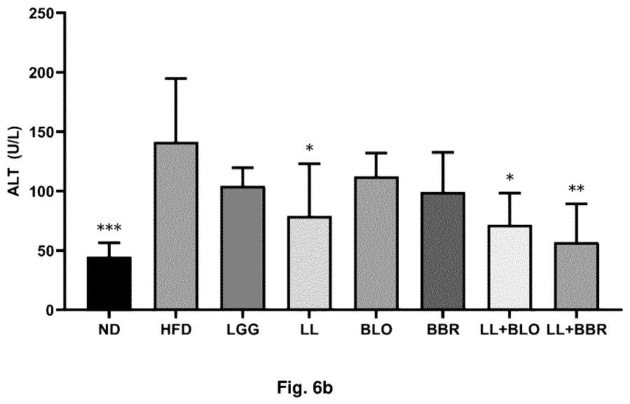

As shown in FIG. 6a, the ALT level was significantly decreased in the CKDB001 single strain administration group of the present invention compared to the western diet-fed group or the LGG-administered group.

In addition, as shown in FIG. 6b, all of the single strain-and composite strain- administered groups has lower ALT levels than that of the western diet-fed group (HFD). Among others, the groups fed with composite strains of LL and Bifidobacterium spp. (LL+BLO and LL+BBR) were measured to decrease in ALT level in a synergistic manner in light of the group singly fed with the strain of the present disclosure.

Therefore, the Lactobacillus lactis CKDB001 strain (LL or CKDB001) of the present disclosure very effective in reducing AST and ALT levels, with the excellent reductive effect on AST and ALT levels in groups fed in combination with Bifidobacterium sp. strains (BLO and BBR).

### Example 4: Inflammatory Cytokines (TNF-alpha, IL-1beta, IL-6)

In order to examine whether the Lactobacillus lactis CKDB001 strain of the present disclosure reduces the mRNA expression and protein expression of inflammatory cytokines in the liver, the following experiment was performed.

RNA was extracted from the liver tissue homogenate prepared by homogenizing the liver tissue of the mice, collected in Example 2, after 9 weeks of the administration of lactic acid bacteria, and cDNA was synthesized therefrom. mRNA expression levels of inflammatory cytokines (TNF-alpha, IL-1beta, IL-6) were determined by comparison with that of GAPDH (housekeeping gene) through qPCR.

Information about products used for PCR analysis is summarized in Table 3.

TABLE 3

| Gene | Product Name | Manufacturer |
|---|---|---|
| TNF-alpha | Mm00443258_m1 | ThermoFisher |
| IL-1beta | Mm00434228_m1 | SCIENTIFIC |
| IL-6 | Mm00446190_m1 | |
| GAPDH | Mm99999915_g1 | |

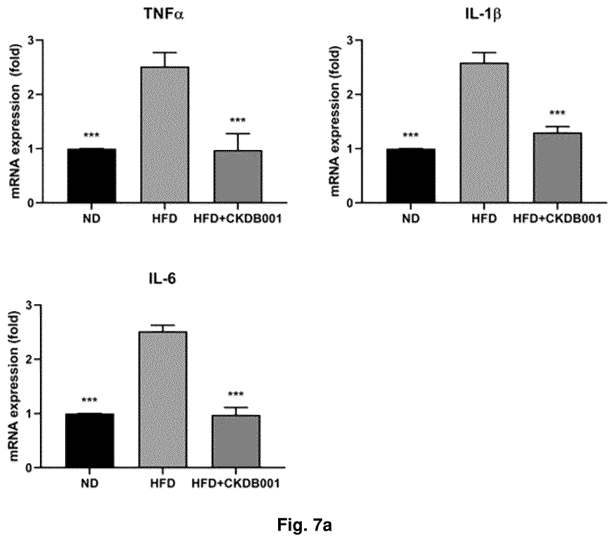

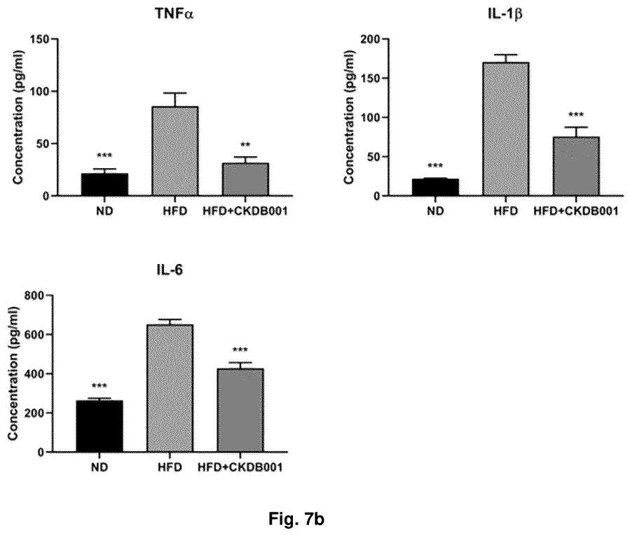

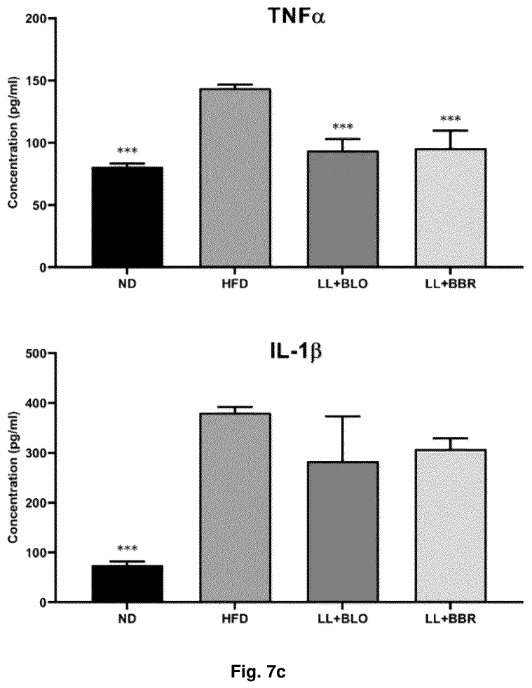

The mRNA expression levels of individual genes in the single strain-administered group are depicted in FIG. 7a.

As shown in FIG. 7a, when the Lactobacillus lactis CKDB001 strain of the present disclosure was administered alone, the mRNA expression levels of inflammatory cytokines, TNF-alpha, IL-1beta, and IL-6 were observed to be significantly reduced similar to that of the normal diet group.

In the following experiments, examination was made to show whether administration of Lactobacillus lactis CKDB001 strain alone or in combination with Bifidobacterium sp. strain reduces the expression of inflammatory cytokines in the liver. After 9 weeks of administration of lactic acid bacteria, liver tissue homogenates prepared by homogenizing liver tissues of the mice, collected in Example 2, were incubated with PRO-PREP™ Protein Extraction Solution (iNtRON Biotechnology, Korea) at 4° C. for 30 minutes and then centrifuged at 10,000 g for 10 minutes. The supernatant thus formed was taken and analyzed for levels of pro-inflammatory cytokines, including tumor necrosis factor (TNF)-α, interleukin (IL)-1beta and IL-6, by enzyme-linked immunosorbent assay (R&D Systems, Minneapolis, MN). The results are depicted in FIGS. 7b and 7c.

Consistent with the mRNA expression levels, as shown in FIG. 7b, the administration of CKDB001 of the present invention significantly reduced the secretion of inflammatory cytokines (TNF-alpha, IL-1beta, and IL-6), compared to the western diet group.

In addition, as shown in FIG. 7c, the secretion of TNF-alpha and IL-1beta was also reduced by administration of the CKDB001 strain of the present invention in combination with Bifidobacterium sp. strains (LL+BLO, LL+BBR).

Therefore, the administration of the Lactobacillus lactis CKDB001 strain of the present disclosure alone and in combination with the Bifidobacterium sp. strains brings about an excellent effect of alleviating western diet-induced non-alcoholic steatohepatitis.

### Example 5: Expression of Tight Junction Protein in Intestinal Epithelial Cells

It is known that feeding with western diet reduces expression of tight junction proteins in intestinal epithelial cells and causes occurrence of leaky gut syndrome. The following experiments were performed to examine the effect of the Lactobacillus lactis CKDB001 strain of the present invention on the expression of tight junction proteins in intestinal epithelial cells.

The intestinal tissue homogenate from the composite strain-administered group

(LL+BLO and LL+BBR) of the present disclosure was incubated with PRO-PREP™ Protein Extraction Solution (iNtRON Biotechnology, Korea) at 4° C. for 30 minutes and then centrifuged at 10,000 g for 10 minutes. The supernatant thus formed was quantitatively analyzed for proteins by Bradford assay, followed by electrophoresis using 10% sodium dodecyl sulfate-polyacryamide gel (SDS-PAGE). Proteins in the polyacryamide gel were transferred to a PVDF (polyvinylidene fluoride) membrane and blocked with 5% skim milk. After washing with TBS-T, the membrane was treated with a rabbit anti-mouse occludin antibody as a primary antibody and then with an HRP-conjugated goat anti-rabbit IgG antibody as a secondary antibody.

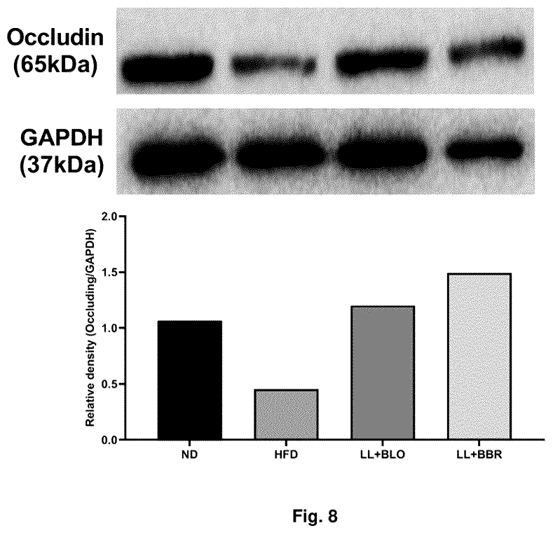

As shown in FIG. 8, the protein expression of occludin in all of the composite strain-administered groups (LL+BLO, LL+BBR) of the present disclosure was similar to or higher than that in the normal diet-fed group.

Therefore, combined administration of the strains was found to remarkably reduce western diet-induced leaky gut syndrome.
Accession Number
Depositary Authority: Korea Research Institute of Bioscience and Biotechnology
Accession Number: KCTC14149BP
Deposition date: 20200303
Depositary Authority: Korea Research Institute of Bioscience and Biotechnology
Accession Number: KCTC13671BP
Deposition date: 20181023
Depositary Authority: Korea Research Institute of Bioscience and Biotechnology
Accession Number: KCTC13893BP
Deposition date: 20190719

1.-8. (canceled)

9. A composition comprising a Lactobacillus delbrueckii subsp. lactis CKDB001 strain with accession number KCTC 14149BP as an active ingredient and a pharmaceutically acceptable carrier.

10. The pharmaceutical composition of claim 9, further comprising a Bifidobacterium sp. strain as an active ingredient.

11. The pharmaceutical composition of claim 10, wherein the Bifidobacterium sp. strain is Bifidobacterium longum, Bifidobacterium breve, or a combination thereof.

12. The pharmaceutical composition of claim 11, wherein the Bifidobacterium longum is Bifidobacterium longum CKDB004 with accession number KCTC 13671BP and the Bifidobacterium breve is Bifidobacterium breve CKDB002 with accession number KCTC 13893BP.
